(19) United States
(12) Patent Application Publication
(10) Pub. No.: US 2021/0233627 A1
(43) Pub. Date: Jul. 29, 2021

(54) CONNECTIVITY PLATFORM TO ACCESS PERSONALIZED LABORATORY DIAGNOSTICS AND RESULTS

(71) Applicant: Peter Foley, Dun Laoghaire (IE)

(72) Inventor: Peter Foley, Dun Laoghaire (IE)

(21) Appl. No.: 16/067,002

(22) PCT Filed: Dec. 30, 2016
(86) PCT No.: PCT/US2016/069418

§ 371 Date: Jun. 28, 2018

(51) Int. Cl. G16H 10/40; G16H 40/67; G16H 10/20; G16H 10/60; G16H 50/20; G16H 80/00

(52) U.S. Cl. CPC G16H 10/40; G16H 40/67; G16H 80/00; G16H 10/60; G16H 50/20; G16H 10/20

(57) ABSTRACT

A system is provided that enables a process centralized from a pharmacy or online portal which allows an individual to access authorized international laboratory tests and results with clinical input to track, monitor and improve their personal health. The system commands a network of global physician portals to approve; review patients requests and support to derive better clinical outcomes. The accounts are linked to anonymized system using patient identifiers and real-time test code assignment to ensure a seamless and confidential exchange of data.

### BACKGROUND OF THE INVENTION

### 1. Field of the Invention

FIG. 1 illustrates the components and use of the invention. A core connectivity platform and a laboratory API are configured to allow a customer to electronically order and manage laboratory results through a secure and personalized online portal.

The invention generally relates to a core connectivity that manages the receipt, transmission, flow and monitoring of analytical data from certified laboratories through a core connectivity platform accessible to consumers.

An individual using the system can obtain credible analytical data from personal health samples through a secure and confidential process facilitated through a laboratory API. Personal and laboratory data are handled through a core system accessed via a secure username and password.

The core connectivity platform acts as an interface between laboratories internationally for personal health diagnostic testing, order and results transmission through an encrypted laboratory API and clinical intervention through a network of connected physicians based on specific specialties. The core platform includes inter alia: transaction and account creation, unique identification and barcode generation, test code assignment to generic tests during test activation, creation of electronic patient records, customer management with clinical and non-clinical expertise, dispatch services through an international center with dedicated APIs, and connectivity with international laboratories with differing specialties for the upload and receipt of test information through a secure laboratory API as part of the core connectivity platform.

### 2. Description of Background Art

Diagnosis and/or detection of many ailments and diseases in humans have taken on added importance, not only for medical diagnosis but also in such areas as illicit drug identification, forensic toxicology and insurance screening. As availability of information concerning diseases is disseminated by social media and the internet, there is increased awareness of the dangers and effects of many heretofore unknown diseases, ailments or cancers and an increased desire to identify and monitor bio indicators and one's own personal health to drive better clinical outcomes through prevention.

As more individuals seek ways to detect, identify and monitor diseases, cancers and other ailments relevant to their own personal health, some companies have developed tests, such as rapid test which can be used with easily obtained body fluid samples. In some instances, the kits provide a means not only to test for the presence of certain materials but also to provide some measure of quantitative levels indicative of diagnosis of a disease or toxic injury.

Part of the general concept of personalized medicine is access by an individual to his own biomarkers, whether these substances are indicators of a specific disease, ailments or provide identification of xenobiotics or other substances which are foreign to the body, such as toxins. Rapid self-testing can be used by non-medically trained persons for preliminary diagnosis or for follow-up medical treatment regimens. While, self-testing kits may provide patients with information about their current health condition they have low sensitivities and specificities thereby resulting in inaccurate results and in some cases, false positives and negative results can occur. Further such tests derive an immediate result and so, there is no support, follow up visit or consultation with a physician. Such tests standalone with no system or support structure.

There are a number of these self-testing kits available for in-store purchase at pharmacies or online. Currently available kits include those for detecting cholesterol, drugs of abuse, and certain indicators of urinary tract infection and pregnancy as well as markers for allergies, anemia, cholesterol, drug abuse, fecal occult blood, hepatitis C, rapid HIV, male infertility, menopause, ovulation prediction, pregnancy, prothrombin time, urinary tract and yeast infections. Several breath-testing kits are currently on the market, including the Easy Sampler Breath Test Kit and Alveo Sampler Breath Test Kit.

Self-sampling collection kits for screening of high-risk STDs allow an individual to collect samples at home and return the sample to a laboratory. MyLabBox is an internet service where an individual can order a kit online, collect a sample using the kit components, mail the collected sample to a designated lab, and receive lab results in a few days via email or by phone call. Several kits are available including a home STD test for HIV, Chlamydia and Gonorrhea and an optional test kit that also includes Trichomonais. The kits arrive in separate components and are sent from different laboratories making the system slow and cumbersome. Samples for these tests are collected from a urine sample and, for some tests, a small blood sample. The test kits are paper based and require a customer to include personal identifiable information on the test and samples. While this allows an individual to avoid a clinic or doctor's office, there can be difficulties in sample collection, proper handling of the sample and questionable interpretation of the results by the individual who typically has no medical training. Further there is no technology platform linking the customer to the test-kit or laboratory, it is simply a mail based system whereby the results are sent to a third party provider who handles the results on behalf of MyLabBox, creating a high probability of error and results assigned to the wrong person.

Self-testing has several advantages, including privacy, convenience, and typically lower cost than services through a physician's office. The FDA classifies at-home kits into two categories: (1) test kits where the individual collects the sample, conducts a test and reads the results being a self-testing kit; and (2) self-sampling collection kits which require sending the collected samples to an outside laboratory.

Unfortunately, self-testing is not ideal. Many self-administered testing kits currently available online are not of high quality. Kits supplied for identification of analytes and/or diagnosis are notoriously inaccurate and are not suitable for initial diagnosis or routine monitoring. Self-testing and diagnosis does not afford the option of a professional assessment of the tests and does not have the advantage of lab results from accredited analytical laboratories.

For self-sampling collection kits whereby samples are mailed to a laboratory, not all laboratories are FDA approved for the types of screening needed for accurate results. Furthermore, similar to a doctor's office such tests require physician input to approve an order before a laboratory can run a test and review results before they can be released to a customer which is rarely the case. Online testing does not address this issue and tests are authorized and approved without a qualified physician. Results are sent via email or returned over the phone by a third party nurse which leads to significant errors from a data and patient management standpoint. The primary issue is the absence of an adequate support structure and the ability to track a disease from a person and provide an adequate support structure should they need treatment and care.

### DETAILED DESCRIPTION OF THE INVENTION

FIG. 1 illustrates the components and use of the invention. A core connectivity platform and a laboratory API are configured to allow a customer to electronically order and manage laboratory results through a secure and personalized online portal. The core connectivity platform links a customer through a personal dashboard to an anonymized test kit which possesses two unique identifiers and a corresponding barcoded system. The system assigns test codes for the different tests to be performed by a laboratory, the tests selected by the customer set up from a dashboard to the test-kit and two unique identifiers within.

All orders and results are transmitted between the core connectivity platform and the laboratory through a dedicated laboratory API. The information flowing through the system is authorized, reviewed and monitored by computer algorithms to determine how the information is presented, managed and distributed throughout the system. There are multiple physician portals to review and authorize laboratory tests and results remotely, whilst providing other clinical portals for physicians, nurses and support staff to interpret and provide guidance to customers through an online consultation and or environment. The platform in essence allows a customer to access, manage and monitor their vitals through laboratory testing, whilst having physician and clinical support through a virtual environment made accessible through a personal online account.

An important aspect of the invention is the core connectivity platform connecting customers to laboratories, physicians, global dispatch, support staff and providing customer access to personalized laboratory testing. Individuals can electronically order laboratory tests through a personalized patient dashboard and use test-kits which are anonymized using an alpha code, numerical code and combined barcode which will be linked to that customer after the fact. The system allows an integrated physician network to authorize and review customer orders and results electronically, before being transferred to the personalized online dashboard.

The system provides a customer access to an online laboratory system. The customer orders a laboratory test online via a secure web application and creates a password protected account. The customer is required to submit the following information for account creation, including first name, second name, date of birth, email address, phone number, which is verified with a text message code that must be resubmitted to proceed, and mailing address. The transaction and payment happens online through the web application.

Once the transaction is completed, the core connectivity platform transmits a secure message to an international dispatch center through a dedicated global dispatch API. The dispatch center sends an anonymized test-kit for sample collection to the customer. The test kit contains no customer information or information regarding what test(s) has been selected by the customer or what analysis is to be performed by the laboratory. The test-kit is generic and contains two unique patient identifiers: an alpha code and a numerical number. The test-kit can be assigned any range of test codes from the core connectivity platform which will happen once the customer receives and activates the test-kit.

The customer's online dashboard provides full traceability of the order and is updated every step of the way: 1) test dispatched; 2) test received by customer; 3) test; 4) awaiting activation; 5) test received by laboratory; and 6) test results available.

The customer is required to activate the test-kit before sample collection. The customer is required to access a personal online account or dashboard and register the unique patient identifiers within the test kit to the personal account. The two identifiers are the alpha code and the numerical code that are provided within the kit. The alpha code and numerical will be visible within the test-kit and are presented as a barcode on the inner lid of the test-kit (see FIG. 2). The unique identifiers are also visible on each of the sample collection devices within the test-kit which may include any one or more of a urine collection device, saliva collection device, capillary testing device, blood collection device, or FIT for stool collection.

When registering the test-kit, the customer may manually enter the unique patient identifiers being the alpha code and numerical code to their personal account or scan the barcode from their mobile or tablet device using the camera which will automatically important the alpha code and numerical code from within the barcode to their account.

The customer will be required to complete a patient medical history and a suitability questionnaire directly after registering the unique identifiers as part of the test-kit activation process. The suitability questionnaire will vary depending on the test type. For example, HIV testing will have a very different criterion from genetic testing. Once the medical history and suitability questionnaire are complete and filed online, the test kit is activated. This suitability questionnaire assists the medial staff in test authorization and when delivering results so that they have a complete picture of the customers health.

By activating the test-kit, the core connectivity platform will link the customer to the two unique identifiers within test-kit. Customer information includes but is not limited to: first name, second, age, contact number (verified), email, complete medical history, suitability questionnaire.

The core connectivity platform will also assign a test code or codes to the unique identifiers within the test-kit based on the customer's order. Until this point the test-kit and identifiers had no pre-defined purpose. The core connectivity platform is responsible for assigning test-codes based on the customer's order and then communicating these test codes to the receiving laboratory. The laboratory will know what tests are to be performed on the test-kit and samples, based on the two unique identifiers and test code(s), before the sample reaches the laboratory.

The customer will then use the test-kit and instructions to collect a sample(s) personally or with assistance and return the sample(s) to a partnering accredited laboratory for analysis. The customer is provided a pre-paid shipping bag and label to return the sample(s) which is fully trackable from within the patient dashboard or online portal. The customer is also provided a patient identification card containing the unique identifiers, should they ever need to activate a test after the fact or should they require a phone call from a member of the medical team. The card helps the care team ensure the customers identity.

After test activation, the electronic patient order will be transferred by the core connectivity platform to an internal physician portal for review and approval. There are physician portals for each country, state and region which are further sub categorized by physician specialty. For instance, oncology, virology, urology and dermatology. The physician will review the entire electronic patient record to include:

Alpha code

Numerical code

First name

Second name

Date of birth

Gender

Medical history

Suitability questionnaire

Test code (identifies test to be performed on the sample).

A physician will review the electronic patient record and exercises his expert medical judgment to determine whether a person is suitable for the testing profile. If the customer is not, the physician can apply notes to the record and he or she may follow up with the customer through the physician portal for more information. This may be achieved via electronic communication or video consultation. If the physician is satisfied with the electronic patient record, it will be marked as approved.

Once approved, the core connectivity platform will transmit the electronic patient record and laboratory request directly to the partnering laboratory using a dedicated API which connects with the laboratories information management system. The laboratory will know that the sample is en route and what tests to perform on the sample before it arrives. An algorithm will identify the appropriate laboratory based on the following criterion: customer country, region or state, test type to be performed. The electronic patient record will be completely anonymized and include the following customer details:

Alpha code (instead of first name)

Numerical code (instead of second name)

Date of birth

Gender

Test code (identifies test to be performed on the sample).

The laboratory will have the electronic patient record and laboratory request electronic patient record before the test has been sent by the customer. This means that the laboratory will know ahead of time; customer demographics, what sample will arrive and what analysis is to be performed by the laboratory upon receipt of the sample.

The method has significant advantages over the steps typically required to have laboratory testing handled through a physician's office or clinic. Traditionally, the laboratory receives a paper laboratory request form with the sample. The paper form specifies the tests a physician would have the laboratory perform. Not only is this an extra step because a customer would need to visit a physician to get the form before dropping a sample into a collection center, but also the form can get lost, get damaged and contains patient identifiable information. The physician must hand write test codes on both the form and the sample. If the form gets lost, damaged or otherwise compromised, the analysis will not be performed. The core connectivity platform and laboratory API makes this process convenient and confidential because all information is anonymized and seamless as all communications are electronic though a laboratory API and require no hand written forms.

The laboratory will receive the test kit and rather than examining a paper form, they will scan the barcode within the test-kit containing the unique identifiers. The laboratory will have already received the electronic patient record and laboratory request from the core connectivity platform post kit activation. As the record will already be on the laboratory system, scanning the barcode will match this record to the test-kit and sample. The laboratory, according to the record, will know exactly what analysis is to be performed, thereby completing removing any errors that may have occurred through legacy systems.

Alpha code (instead of first name)

Numerical code (instead of second name)

Date of birth

Gender

Test code (identifies test to be performed on the sample).

The laboratory will perform analysis and return the results through a secure laboratory API. The core connectivity platform will identify the unique patient include the alpha code and numerical code and match the result file to a customer personal account.

The laboratory API provides a much more seamless and scalable solution for laboratories, because the requirement to have paper forms is removed and the system is entirely digital.

The core connectivity platform uses an algorithm to triage the results file and determine how the result should be handled. In the first instance, result files are transferred to the physician portal, whereby the original electronic patient record was approved. The physician will have the ability to review the entire patient record, associated results and assign medical notes and advice to the electronic patient record and results. The physician marks the file as approved and the record and results are transferred directly to the customer's electronic dashboard or to another clinician portal depending on the severity of the result or if a certain specialty is required for patient management.

The core connectivity platform can determine whether a result needs further clinical intervention or not. For example, if a numerical result is in range or a qualitative result defined as “not detected” or “negative” the result will be transmitted directly to the customer's dashboard. The customer will receive a notification via text message, email or push notification by mobile app. The customer will login using a username, password and review results.

The core connectivity platform can also use an algorithm to determine the severity of the result and assign the result to a specialized nursing support dashboard. For instance, if the results are related to sexual health, a sexual health nurse will be issued the electronic patient record to manage the patient and result. If the result pertains to oncology, fertility or genetics, a different nursing or specialist portal is notified. The specialist nurse or physician will contact the customer by phone or video call into the customer's dashboard or personal account. The customer will be provided clinical support, e-treatment or prescribing or a referral pathway into the clinical setting.

The system also allows a specialist nurse or physician to e-prescribe or provide an electronic prescription should a customer require treatment or care. For example, in the case of a positive sexually transmitted infection the customer may require an antibiotic to treat an infection. The assigned nurse or physician can access an e-prescribing option from the clinician portal. A series of questions will be asked to determine a customer's suitability for the e-prescription. In some instances, a referral pathway into the clinical setting may be deemed more appropriate. The physician or nurse will ask a number of pre-defined questions and add additional comments which will be added to the customer electronic patient record. If a prescription is appropriate, a summary of the consultation will be transmitted to the customer's dashboard where further consent will be required. The customer will review and agree to the terms and conditions applicable to the e-prescription. The customer will also have the ability to determine the pharmacy in which the e-prescription will be sent either verbally when specking with the nurse or physician or personally through the customer dashboard. Both portals will have the capability of selecting the correct pharmacy, as the customer may not know during the consultation and choose after the fact via the personalized dashboard.

The electronic patient record will be stored for each customer in the core connectivity platform and reflect all orders, unique identifiers applicable to order and test, medial history, suitability questionnaires, nurse calls, physician voice calls and video calls, e-prescriptions and antibiotics used and all information pertaining to the customer and will be accessible to the customers as a going concern for use as an electronic patient medical record.

The core connectivity platform provides a diagnostic and patient management solution for pharmacies, with real-time physician laboratory request authorization and clinical support for its customers in need of convenient and accessible laboratory testing, treatment and care. Pharmacies have a turnkey diagnostics solution to perform laboratory tests without the need to have a physician or clinical support team in the consultation suite at all times.

A customer will have the ability to access a range of laboratory tests through a dedicated portal located within the consultation suite of a pharmacy or in-store. Unlike the online model, the transaction, account creation, test registration and activation, suitability questionnaire and remote physician approval happens in real-time through a dedicated web application or browser with the pharmacy consultation suite or in-store.

To access laboratory testing, a customer will access a pharmacy consultation suite or area within the pharmacy and can choose from a suite of tests through a web application accessible through a dedicated tablet device or browser. The process can be completed with the assistance of a pharmacist or a member of the pharmacy team. The transaction and payment happens through the web application in-store.

The customer will select a laboratory test(s) and create a password protected account. The customer is required to submit the following information for account creation: first name, second name, date of birth, email address, phone number (which is verified with a text message code that must be resubmitted to proceed) and mailing address.

Once the account has been created, the customer will be prompted and supplied a generic test-kit containing 2 unique identifiers: alpha code and numerical code. This test will have no function or pre-determined classification until linked to the customer and a test code has been assigned by the core connectivity platform.

A pharmacy or retail partner has the ability to assign test-codes to a generic test-kit depending on a customer's order. In essence the test-kit can perform any function once assigned the relevant test code. The customer will activate the test by registering the unique identifiers to the personal dashboard or web portal. The customer will be required to complete a medical history and suitability questionnaire as part of the kit activation process.

Once activated, the customer is linked to the test-kit. The customer's details and electronic patient record are linked to the test-kit and the identifiers within. The core connectivity platform will assign a test-code, thereby providing the test-kit and accompanying identifiers with a purpose or function. The laboratory will now perform the appropriate tests for this particular test-kit and sample(s) once received.

As described, the electronic patient record and order will be transferred by the core connectivity platform to an internal physician portal for review and approval. There are physician portals for each country, state and region which are further sub categorized by physician specialty. For instance, oncology, virology, urology and dermatology. The physician will review the entire electronic patient record to include:

Alpha code

Numerical code

First name

Second name

Date of birth

Gender

Medical history

Suitability questionnaire

Test code (identifies test to be performed on the sample).

A physician will review the electronic patient record and exercise his expert medical judgment to determine whether a person is suitable for the testing profile; if not, the physician can apply notes to the record and may speak with the customer through a video consultation via the customers dashboard. If the physician is satisfied with the electronic patient record, it will be marked as approved. This process will happen in real-time from the pharmacy and through the customers personalized dashboard or web portal which is accessible through the web application available within the pharmacy.

Depending on the type of test required, the customer may elect to take the test home for self-collection or request assistance from a pharmacist, technician, phlebotomist or whomever is present within the consultation suite. As with the online model, each test is supplied with a return shipping label to ensure delivery to the relevant laboratory partner.

Once approved by the physician, the core connectivity platform transmits the electronic patient record and order request directly to the partnering laboratory using a dedicated API which connects with the laboratories information management system. The laboratory will know that the sample is en route and what tests are to be performed on the sample before it arrives. An algorithm will identify the appropriate laboratory based on the following criterion: customer country, region or state, test type to be performed. The electronic patient record will be completely anonymized and include the following customer details:

Alpha code (instead of first name)

Numerical code (instead of second name)

Date of birth

Gender

Test code (identifies test to be performed on the sample).

The laboratory will have the electronic patient record and order request on the their system before the customer has sent back the sample. This means that the laboratory will know ahead of time; customer demographics, what sample will arrive and what analysis is to be performed by the laboratory upon receipt of the sample.

The laboratory receives the test kit and will scan the barcode within the test-kit containing the unique identifiers. As stated, the laboratory will have already received the electronic patient record and order from the core connectivity platform following kit activation and physician approval. As the record will already be on the laboratory system, scanning the barcode will match this record to the test-kit and sample. The laboratory, according to the record received, is informed what analysis is to be performed, thereby completing removing any errors that may have occurred through legacy systems.

Alpha code (instead of first name)

Numerical code (instead of second name)

Date of birth

Gender

Test code (identifies test to be performed on the sample).

The laboratory performs analysis and returns the results through a secure laboratory API. The core connectivity platform identifies the unique patient identifiers and matches the result file to a customer.

The laboratory API provides a much more seamless and scalable solution for laboratories, because the requirement to have paper forms is removed and the system is entirely digital.

The core connectivity platform uses an algorithm to triage the results file and determines how the result is handled. In the first instance, result files will be transferred to the physician portal, whereby the original electronic patient record was approved. The physician will have the ability to review the entire patient record, associated results and assign medical notes and advice to the electronic patient record and results. The physician then marks the file as approved and the record and results are transferred directly to the customer's electronic dashboard or to another clinician portal depending on the severity of the result or if a certain specialty is required for patient management.

The core connectivity platform will also use an algorithm to determine the severity of the result and assign the result to a specialized nursing support dashboard. For instance, if the results are related to sexual health, a sexual health nurse will be issued the electronic patient record to manage the patient and result. If the result pertained to oncology, fertility or genetics, a different nursing or specialist portal would be notified. The specialist nurse or physician will contact the customer by phone or video call into the customer's dashboard or personal account. The customer will be provided clinical support, e-treatment or prescribing or a referral pathway into the clinical setting.

If a numerical result is in range or a qualitative result defined as “not detected” or “negative”, the information will be transmitted directly to the customer's dashboard. The customer will receive a notification via text message, email or push notification by mobile app. The customer will login using a username, password and review results.

The pharmacist will not be required to provide further input once the customer leaves the pharmacy. The customer will be managed in all instances through their personal online dashboard and core connectivity platform which provides clinical and nursing support for those in need of support, care or treatment. All retail partners are provided a remote diagnostics solution without the need for developing a patient management system or developing a physician network or clinical support structure themselves.

The electronic patient record will be stored for each customer in the core connectivity platform and reflect all orders, unique identifiers applicable to order and test, medial history, suitability questionnaires, nurse calls, physician voice calls and video calls, e-prescriptions and antibiotics used and all information pertaining to the customer and will be accessible for the customers as a going concern for use as an electronic patient medical record.

### BRIEF SUMMARY OF THE INVENTION

The present invention addresses many, if not all, of the disadvantages inherent in currently available testing and the steps involved in obtaining confidential, personal, quality analytical results. Unlike traditional tests which are available online or through a pharmacy, the core connectivity platform provides a more accurate diagnostics solution through accredited laboratory testing, with real-time physician approval and clinical support through a customer's personal online dashboard.

The disclosed system allows an individual to access laboratory testing online or within a professional, entirely confidential environment without requiring a physical doctor's visit or an exam. Confidential results are provided directly to the individual with a reduced probability of false negatives or false positives due to the accuracy of laboratory results, whilst providing physician authorization and clinical support via a secure and personalized web portal.

The system is designed to interpret test analysis for a range of tests to include urine, blood, stool and saliva samples efficiently, accurately and in confidence. The core connectivity platform assigns test codes to anonymized test kits containing unique patient identifiers to include alpha codes and numerical codes. The core connectivity platform transmits approved orders and receives results through a laboratory API. The core connectivity platform uses an interpretation algorithm to triage the results and present them in a manner which is understandable for a customer. An overview of the core connectivity platform is shown in FIG. 1.

The process is initiated by a customer seeking to access a laboratory test online or through the consultation suite of a pharmacy or through a dedicated area within a pharmacy and through the dedicated web application and patient portal. The customer may choose to create an account online or through the convenience of a pharmacy consultation whereby additional support and a brief consultation may be provided. The customer will select a test, create a secure personalized online account and transact through a dedicated web portal in the pharmacy.

The test-kit provides means for collecting samples, packaging and instructions for sending to a designated certified analytical laboratory, a unique personal identification number (alphanumeric code) for registration to the customer's account and directions for sample collection.

Pharmacies are an important aspect of the invention system. Pharmacies consultation suites or designated spaces in pharmacies are extremely accessible and not only assist in setting up confidential personalized web accounts with the core connectivity platform but also provide immediate assistance and access to the individuals who wants to have confidential, high-quality testing for a wide range of analytes, including sexually transmitted infections or diseases, cancer screening to include prostate cancer, bowel cancer, cervical cancer and BRCA gene mutation for breast cancer, other diseases such as diabetes, cardiovascular disease, testosterone, iron, vitamin, thyroid, fertility testing or other markers as available from accredited laboratories. It is not necessary to visit a physician or clinic to have professional assistance in collecting samples such as blood, urine, sweat or saliva nor is it necessary to have a physician physically present to authorize the laboratory request. These physical constraints are handled electronically and virtually via the dedicated web portal and customer's online dashboard. The visit to the pharmacy suite is similar in some ways to using a walk-in or immediate care clinic except that no doctor or medical professional is physically present. The pharmacy suite or dedicated area is set up to assist the individual in accessing the system connected with use of the purchased self-sampling or pharmacy assisted tests. Furthermore, the core connectivity platform provides access to a team of qualified medical professionals, including physicians to authorize lab tests and provide online consultations through the personalized web portal.

A pharmacist on duty acts as a professional assistant to the extent required and assists a customer with selecting the most appropriate test via a tablet device or dedicated web portal within the consultation suite of the pharmacy or in store, to create a personal account for the customer through the core connectivity laboratory platform with username and password, to supply a generic test which is pre-assigned unique identifier and activate the test by registering the alpha code and numerical code to the customers personal account, complete the medical history and suitability questionnaire linked to the personal account, wait for real-time physician approval, offer to assist the customer in collecting the sample(s) or advise that this may be completed on the customers own time and explain the sample mail and results delivery process via the customers personal online account.

Once activated, the LGC core connectivity platform will assign the relevant test codes and customer information to the alpha code and numerical codes within the test-kit and transmit this patient information in the form of an electronic patient record to an LGC physician portal for immediate approval and authorization. This will happen in real-time while the customer is in the pharmacy consultation suite and upon approval by the physician will send an electronic communication to the receiving laboratory to notify them that the sample is in transit and what analysis is to be performed on the sample when it arrives.

Pharmacies benefit from a countless number of testing profiles via the dedicated web portal and core connectivity platform; which customers can avail of Rather than having stock which is branded with different sleeves, branding and product codes, there is a single generic test-kit which is assigned a purpose or test codes during kit activation. The customer can assign any test code, to a single kit through the web application located within the consultation suite of the pharmacy.

The pharmacist provides consultation regarding any aspect of the mail-in process but not in any capacity as a medical doctor. No record of an individual name or personal information is taken with respect to the purchase and is all stored within the customers personal online account which is created within the consultation suite.

Real time physician approval is an important aspect of the core connectivity platform enabling a customer to access real-time laboratory testing online or through a pharmacy location, with remote physician approval/authorization. After test-kit activation, a physician will access the order, medical history and suitability questionnaire to determine if the customer is suitable for the particular test. The physician will have the ability to engage the customer through an electronic or video consultation if further dialogue is required. This removes the necessity for a customer to obtain a laboratory request from a physician's office and then having to visit a laboratory drop-in center to have the test performed or sample collected. This also provides pharmacies or retail providers with the ability to deploy diagnostic testing with remote physician approval/authorization of laboratory orders and results through a core connectivity platform.

Online orders follow a similar process; only the account creation process takes place from the customer directly through a mobile device. The customer chooses a tests, creates a personal account and completes payment online. Online orders are processed and dispatched from the global dispatch center which has a dedicated API for connectivity to the core platform (FIG. 1).

The customer, upon receipt, is required to activate the anonymized test by registering the an alpha code and numerical code provided in the test (FIG. 2) to their personal online account. The customer is required to complete a medical history and suitability questionnaire linked to the order. The core connectivity platform assigns a test code to the alpha and numerical code within the test-kit.

As per the pharmacy flow, an electronic patient record will be sent to the relevant physician portal for review and approval. Once approved the core connectivity platform sends the electronic patient record to the laboratory containing basic patient demographics, the alpha code, numerical code and test codes to be performed by the laboratory through a dedicated API.

Privacy, confidentiality, efficiency and confidence in a laboratory analysis are important to an individual seeking either one-time or periodic monitoring of analytes such as sexually transmitted infections, genetic, cancer, fertility or lifestyle screening for specific health markers such as cholesterol, disease markers and the like.

An important component of the system is the provision of unique identification codes which possess an alpha code and a numeric code which are pre-accessioned to each of the self-sampling test kits prior to a customer receiving them, either online or through a pharmacy. These codes are linked a customer during the account creation process and ensure that the test kit and subsequent results are confidential, anonymized and transmitted only to the individual. The test-kits are also generic and have no pre-defined purpose until the core connectivity platform assigns them a test code and purpose. This means the test-kits are unique and can perform any task, as commanded by the customer and assigned by the core connectivity platform.

Irrespective of whether the system was initiated online or within a pharmacy, the laboratory will receive an electronic patient record and order, once the test has been authorized by a physician. The record will include the alpha code, numerical code and test code to be performed by the laboratory. This record will rest in the laboratory system until the samples arrive. When the samples arrive, the laboratory will scan the barcode within the kit and the electronic record is pulled from their system. The record and test-kit are matched and the analysis is performed as per the electronic patient record and order which includes the test codes as previously assigned by the core connectivity platform and communicated via the encrypted laboratory API.

Once analysis has been completed, the laboratory API will receive results from the laboratory through an encrypted communication and use an algorithm to interpret qualitative and quantitative laboratory diagnostic results and determine the appropriate physician or nursing dashboard to handle the case. The test type, result type and location of the customer will all be deciding factors. The core connectivity platform will also interpret qualitative and quantitative results and present the outputs into a meaningful, understandable and presentable fashion for both the physicians and customers alike. The raw data from the laboratory, will be pushed to the electronic dashboard and manipulated in such a way that is understandable and supporting narratives will be generated to accompany the results which will be derived from a complex algorithm based on result ranges and customer demographics.

The system also includes an electronic e-prescribing software tool which allows a clinician via a web portal to access the customer's electronic patient medical record, complete a suitability questionnaire for the assignment of antibiotics and electronically transfer the said e-prescription to a designated pharmacy for collection by the customer. The physician or nurse will ask the customer a range of questions, defined as a prescribing suitability questionnaire, reevaluate the medical history. This will happen verbally or via a video consultation. The physician or nurse will complete the questionnaire, which will be applied to the customer's electronic patient record. The physician or nurse may select a pharmacy or the customer may do so via a map located on the personalized dashboard. The prescription will be electronically transferred to the partnering pharmacy and the medication may be collected by the customer.

The core connectivity platform acts as an interface for customers and facilitates connectivity between laboratories internationally for testing, results transmission and clinical intervention through a network of connected physicians based on specific specialties. The core platform includes, inter alia, the following processes as illustrated in FIG. 1: test selection online or through a pharmacy, transaction and account creation, unique identifiers and barcode generation, assignment of test codes and patient information to unique identifiers within a test-kit, self-collection kits and other kits for a range of sample collection (urine, blood, saliva, swab and stool), physician and nursing portals to approve electronic patient orders and results electronically, customer management with clinical and non-clinical expertise, dispatch services through an international center with dedicated APIs, and connectivity with international laboratories with differing specialties for the transmission of orders and receipt of test information through a secure laboratory API as part of the core platform. Another unique aspect of the system is a software developed algorithm to interpret laboratory diagnostic reports, read and interpret said reports, triage said reports and push to the relevant global physician or nursing portal, release and present laboratory reports (qualitative and quantitative) in a manner that is understandable to a customer and allow them to tack and monitor results over-time. The core interpreter. When company test kits and core connectivity platform is utilized as referenced herein, an individual can enjoy the advantages of the system.

The core platform includes inter alia: transaction and account creation, test selection online or through a remote location, anonymized product and barcode generation, the assignment of electronic patient records and test codes to unique identifiers, laboratory API for the transmission of order requests and results through secure HL7 messaging system, physician portals to approve orders electronically, physician dashboards to review patient records electronically, intelligence software to read, interpret and manage results, nursing dashboards to review patient records electronically, dashboards for other clinical and non-clinical users to engage and manage customers, monitoring and interpretation of analytical data from a certified laboratory and dispatch services through an international center with dedicated APIs, and connectivity with international laboratories with differing specialties for the upload and receipt of test information through a secure laboratory API as part of the core platform. An individual will have the data transmitted to their personalized dashboard whereby they can manage and control their laboratory results.

Laboratory API for transmission of laboratory results: Sending anonymized and electronic communication to and from a laboratory, with test codes and results linked to a unique set of patient identifiers: 6-digit alpha code and 12-digit numerical code through a dedicated laboratory API being the core connectivity platform.

Assigning test codes to a test-kit: Assigning patient information and test codes (test to be performed by a laboratory) to a generic test-kit containing a unique set of identifiers to include a 6-digit alpha code and 12-digit numerical code either online or via a web application within a pharmacy. Customers can access an any tests and the core connectivity platform will assign the codes to the test kit after the fact. The core connectivity platform and the receiving laboratory will know what tests to be performed on the sample (blood, urine, saliva, stool) upon receipt of the sample. The said alpha codes, numerical codes and electronic patient record are sent via the core connectivity platform via a laboratory API to a partnering global laboratory ahead of time and so that the laboratory know what test is to be performed, before the test arrives.

Pharmacies can deliver a countless tests to customers through this system and by using a single test-kit. Rather than having stock which is branded with different sleeves, branding and product codes. The pharmacist can assign any test code, to a single kit through the web application located within the consultation suite of the said pharmacy.

Real time physician approval: An important aspect and invention of the core connectivity platform is the ability for a customer to access real-time laboratory testing online or through a pharmacy location, with remote physician approval/authorization. During test-kit activation, a physician will access the order, medical history and suitability questionnaire to determine if the customer is suitable for the particular test. The physician will have the ability to engage the customer through an electronic or video consultation if further dialogue is required. This removes the necessity for a customer to obtain a laboratory request from a physician's office and then having to visit a laboratory drop-in center to have the test performed or sample collected. This also provides pharmacies or retail providers with the ability to deploy diagnostic testing with remote physician approval/authorization of laboratory orders and results through a core connectivity platform.

Global connectivity of clinical support: Creating global connectivity for physician approval of orders and results through dedicated portals across multiple markets, with clinical and nursing support either through the consultation suite of a pharmacy or online or through a video consultation and electronic approval.

The core interpreter: A software developed algorithm to interpret laboratory diagnostic reports, read and interpret said reports, triage said reports and push to the relevant global physician or nursing portal, release and present laboratory reports (qualitative and quantitative) in an manner that is understandable to a customer and allow them to tack and monitor results over-time. The core interpreter.

E-prescribing: An electronic e-prescribing software tool which allows a clinician via a web portal to access the customer's electronic patient medical record, complete a suitability questionnaire for the assignment of antibiotics and electronically transfer the said e-prescription to a designated pharmacy for collection by the customer.

### BRIEF DESCRIPTION OF THE DRAWINGS

FIG. 1 is a platform overview showing the system and process used for collecting and delivering urine or blood samples to a designated laboratory, and transmission of test results to the individual.

FIG. 2 shows an exemplary sticker which includes the pre-assigned alpha and numerical codes which are system generated and assigned to a test-kit. The test kit remains generic and is only assigned a test code by the core connectivity platform once the kit has been activated which will correspond to the customer's order.

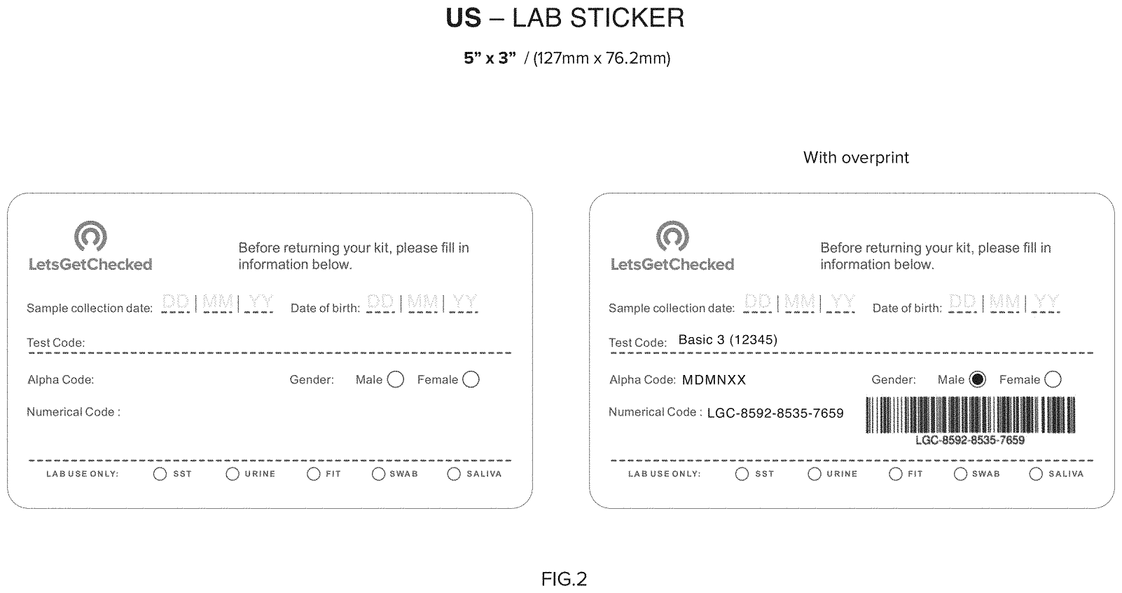

FIG. 3 shows an exemplary patient card that can be retained by the customer for patient identification purposes relative to an order. The patient card contains the alpha and numerical code assigned to the test-kit.

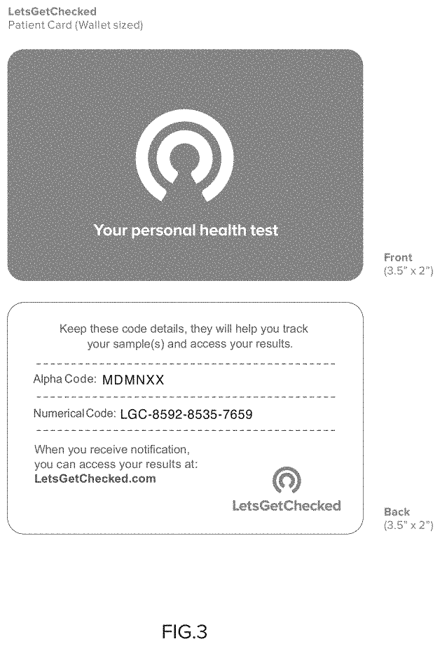

### EXAMPLES

The following examples are provided as illustrations of the invention and are in no way to be considered limiting.

### Example 1

A customer orders online or purchases a company test kit at a consultation suite of a pharmacy. The test kits are completely anonymized and carry no personal or sensitive information pertaining to the customer.

Online orders are dispatched via a global dispatch center directly to the customer whereas pharmacy test kits are available within the pharmacy consultation suite at the point of purchase, account creation and kit activation. The customer creates a personal account during the purchase process (password and username) and provides personal information for the creation of an electronic request form for transmission from the core company platform to the laboratory via a dedicated API.

As part of the account creation process, a customer registers a pre-accessioned test kit to the personal account using the alpha and numerical code provided in the test kit. The customer's account is then linked to the anonymized test kit and a test code is assigned by the core connectivity platform to the test-kit. The electronic patient record is transmitted to a physician portal for real-time approval and further consultation if required. If approved, the electronic patient request is transmitted via an encrypted communication from the core connectivity platform and through the laboratory API. The record will be anonymized and contain the alpha code, numerical code and test code to be performed by the laboratory.

The customer uses clear instructions to collect a blood, urine or saliva sample depending on the type of test selected. The samples can also be collected with the assistance of a professional or assistant depending on the environment. Samples cover inter alia: sexual health, cancer screening, genetic screening, lifestyle screening and fertility testing.

The customer places the samples into a protective box to comply with shipping regulations and then places the box in a pre-paid, self-addressed envelope and mails to the laboratory for analysis. The laboratory will have already received the electronic patient record and know what analysis is to be performed against the specific patient identifier before the sample arrives.

Upon receipt of the samples, scan the test kit barcode and match the sample to the patient record previously received. The laboratory will provide confirmation back though the laboratory API to the core connectivity platform that the sample has been received. The interpreter will use this communication and update the customer dashboard to “sample received by laboratory”.

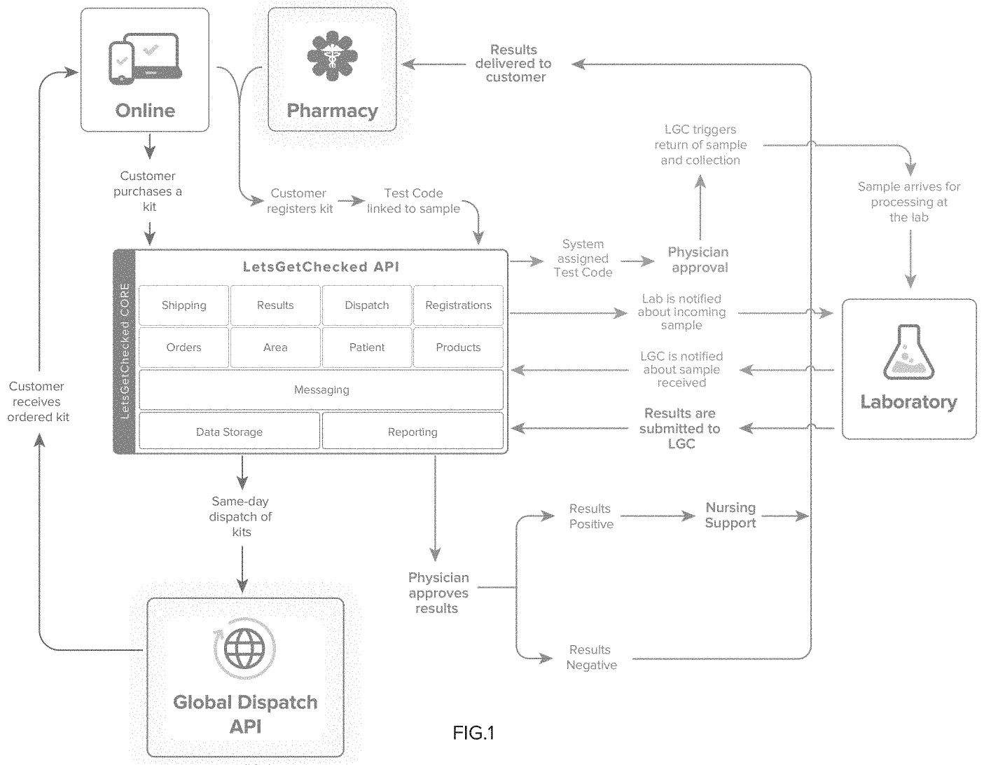

The laboratory will complete the analysis, as per the test code transmitted and return result to the core connectivity platform based on the electronic patient record and unique identifier contained within (see FIG. 1). Results are released to the customer's account and are accessible through a secure mobile platform.

For positive results or results requiring clinical intervention, a doctor or nurse will access a dedicated dashboard and contact the customer directly to communicate the test information. The nurse or doctor will provide support or a patient pathway into the clinical setting for treatment or provide an e-prescribing option. If a genetic predisposition is identified (bowel cancer or cardiac) the system is capable of delivering subsequent tests to monitor the issue (e.g., blood glucose, lipid profile, cholesterol, and stool samples).

### Example 2. Rapid Confidential Analysis of Samples for Sexually Transmitted Infections or Cancer Screening

A customer visits the consultation suite of a pharmacy to get emergency contraception, at which point a sexual health screen is offered providing an immediate link sale. Alternatively, a customer uses a pharmacy locator (map) to find a participating consultation suite. The pharmacist assists the customer in choosing a test and creating a personal account that includes the following information: name, date of birth, sex, sexual orientation, nationality, county, country, location of pharmacy. The pharmacist assists with kit activation using the unique patient identifier, completion of the medical history and suitability questionnaire linked to the particular test via the dedicated web portal.

The customer uses clear instructions to collect a blood, urine or saliva sample depending on the type of test selected. The samples can also be collected with the assistance of a professional or assistant depending on the environment. Samples cover inter alia: sexual health, cancer screening, genetic screening, lifestyle screening and fertility testing.

The customer places the samples into a protective box to comply with shipping regulations and then places the box in a pre-paid, self-addressed envelope and mails to the laboratory for analysis. The laboratory will have already received the electronic patient record and know what analysis is to be performed against the specific patient identifier before the sample arrives.

Upon receipt of the samples, scan the test kit barcode and match the sample to the patient record previously received. The laboratory will provide confirmation back though the laboratory API to the core connectivity platform that the sample has been received. The interpreter will use this communication and update the customer dashboard to “sample received by laboratory”.

The laboratory will complete the analysis, as per the test code transmitted and return result to the core connectivity platform based on the electronic patient record and unique identifier contained within (see FIG. 1). Results are released to the customer's account and are accessible through a secure mobile platform.

For positive results or results requiring clinical intervention, a doctor or nurse will access a dedicated dashboard and contact the customer directly to communicate the test information. The nurse or doctor will provide support or a patient pathway into the clinical setting for treatment or provide an e-prescribing option. If a genetic predisposition is identified (bowel cancer or cardiac) the system is capable of delivering subsequent tests to monitor the issue (e.g., blood glucose, lipid profile, cholesterol, and stool samples).

If the result is in range or a qualitative result is defined as negative, not detected and so on, the results are transmitted directly to the customer dashboard or online portal. The customer receives a text message, email notification or push notification vi a mobile app once the results become available and can login and view them, The customer may seek non-clinical intervention via a nurse call from the personalized dashboard or return to the pharmacy to discuss possible dietary or lifestyle options.

1-20. (canceled)

21. A system for providing remote laboratory testing and remote medical diagnosis, the system comprising:
a core connectivity platform in communication with at least one of an online portal for use by a customer, at least one medical laboratory located remote from the customer, and at least one physician portal for use by a corresponding at least one medical professional located remote from the customer; and
an application program interface (API) in communication with and operatively coupled to the core connectivity platform, the core connectivity platform and API adapted to transmit data to and receive data from at least one of the customers, the at least one medical laboratory, and the at least one medical professional, wherein at least one of the core connectivity platform and the API:
receives a request for at least one laboratory test from the customer using the online portal;
receives authorization for each of the requested at least one laboratory test from at least one first medical professional using a first physician portal;
alerts the at least one medical laboratory of the at least one authorized laboratory test from the customer;
receives results of the at least one authorized laboratory test from the at least one medical laboratory; and
facilitates at least one second medical professional interpreting the results and providing, using a second physician portal, medical guidance based thereon to the customer.

22. The system of claim 21, wherein the API is further operatively coupled to a global dispatch center and adapted to transmit data to and receive data from the global dispatch center.

23. The system of claim 22, wherein, after receiving the request for the at least one laboratory test, at least one of the core connectivity platform and the API further requests the global dispatch center to deliver a sample collection test kit to the customer for each of the at least one requested laboratory test.

24. The system of claim 23, wherein the delivered sample collection test kit is agnostic as to the at least one laboratory test to be performed on a collected sample.

25. The system of claim 23, wherein the delivered sample collection test kit is agnostic as to an identity of the customer.

26. The system of claim 23, wherein the delivered sample collection test kit comprises a generic, anonymized test kit.

27. The system of claim 23, wherein the delivered sample collection test kit comprises two unique identifiers.

28. The system of claim 27, wherein the two unique identifiers comprise an alpha code and a numerical code that, in combination, identify the customer.

29. The system of claim 27, wherein alerting the at least one medical laboratory of the at least one authorized laboratory test comprises providing the at least one medical laboratory with the two unique identifiers of the delivered sample collection test kit.

30. The system of claim 23, wherein the delivered sample collection test kit comprises at least one barcode.

31. The system of claim 30, wherein the at least one barcode comprises an alpha code and a numerical code that, in combination, identify the customer.

32. The system of claim 30, wherein alerting the at least one medical laboratory of the at least one authorized laboratory test comprises providing the at least one medical laboratory with the at least one barcode of the delivered sample collection test kit.

33. The system of claim 30, wherein the at least one barcode identifies the at least one laboratory test to be performed on a collected sample returned with the sample collection test kit.

34. The system of claim 21, wherein receiving authorization from the at least one first medical professional comprises assigning at least one test code to each authorized laboratory test.

35. The system of claim 21, wherein when receiving authorization from the at least one first medical professional at least one of the core connectivity platform and the API further:
identifies a first medical professional to authorize the at least one laboratory test; and
transmits the request and some portion of a medical history of the customer to the identified first medical professional.

36. The system of claim 21, wherein at least one of the core connectivity platform and the API further at least one of:
receives confirmation that the customer has registered the sample collection test kit in a customer account;
receives confirmation that the customer has registered two unique identifiers associated with the sample collection test kit in the customer account;
receives confirmation that the customer has activated the sample collection test kit;
receives confirmation of the at least one laboratory test requested by the customer;
and
receives confirmation that the at least one medical laboratory has received the activated sample collection test kit.

37. The system of claim 36, wherein, when receiving confirmation that the customer has activated the sample collection test kit, at least one of the core connectivity platform and the API further at least one of:
receives patient medical history data from the customer; and
receives a suitability questionnaire completed by the customer.

38. The system of claim 36, wherein, after receiving confirmation that the customer has activated the sample collection test kit, at least one of the core connectivity platform and the API further attributes the customer to the registered two unique identifiers.

39. The system of claim 36, wherein, after receiving confirmation that the customer has activated the sample collection test kit, at least one of the core connectivity platform and the API further attributes the customer to an assigned test code.

40. The system of claim 21, wherein at least one of the core connectivity platform and the API further:
assigns a test code to each authorized sample collection test kit that has been delivered to the customer; and
matches laboratory test results with the customer using the assigned test code.

41. The system of claim 21, wherein, when facilitating the at least one second medical professional interpreting the results, at least one of the core connectivity platform and the API further transmits the results of the at least one laboratory test to the at least one second medical professional.

42. The system of claim 21, wherein, when facilitating the at least one second medical professional providing medical guidance based thereon, at least one of the core connectivity platform and the API further transmits at least one of medical records of the customer, personal medical history of the customer, and at least one suitability questionnaire completed by the customer to the at least one second medical professional.

43. The system of claim 21, wherein, when facilitating the at least one second medical professional providing medical guidance based thereon, at least one of the core connectivity platform and the API further at least one of:
enables the at least one second medical professional to communicate with the customer via at least one of a video conference via the online portal of the customer, a telephone conference via a customer's telephone, and a cellphone conference via a customer's cellphone; and
enables the at least one second medical professional to transmit to the online portal of the customer at least one of: laboratory test results, an e-prescription, medical notes of the medical professional, and medical advice of the medical professional.

44. A method of providing remote laboratory testing and remote medical diagnosis using a system comprising a core connectivity platform in communication with at least one of an online portal for use by a customer, at least one medical laboratory located remote from the customer, and at least one physician portal for use by a corresponding at least one medical professional located remote from the customer; and an application program interface (API) in communication with and operatively coupled to the core connectivity platform, the core connectivity platform and API adapted to transmit data to and receive data from at least one of the customer, the at least one medical laboratory, and the at least one medical professional, the method comprising:
receiving a request for at least one laboratory test from the customer using the online portal;
receiving authorization for each of the at least one requested laboratory test from at least one first medical professional using a first physician portal;
alerting the at least one medical laboratory of the at least one authorized laboratory test from the customer;
receiving results of the at least one authorized laboratory test from the at least one medical laboratory; and
facilitating at least one second medical professional interpreting the results and providing, using a second physician portal, medical guidance based thereon to the customer.

45. The method of claim 44, wherein the API of the system is further operatively coupled to a global dispatch center, the method further comprising:
transmitting data to and receiving data from the global dispatch center; and
after receiving the request for the at least one laboratory test, requesting the global dispatch center to deliver a sample collection test kit to the customer for each of the at least one requested laboratory test.

46. The method of claim 45 further comprising assigning two unique identifiers to the delivered sample collection test kit.

47. The method of claim 46, wherein the two unique identifiers comprise an alpha code and a numerical code that, in combination, identify the customer.

48. The method of claim 46, wherein alerting the at least one medical laboratory of the at least one authorized laboratory test comprises providing the at least one medical laboratory with the two unique identifiers of the delivered sample collection test kit.

49. The method of claim 44 further comprising assigning at least one barcode to the delivered sample collection test kit.

50. The method of claim 49, wherein the at least one barcode identifies the at least one laboratory test to be performed on a collected sample returned with the sample collection test kit.

51. The method of claim 49, wherein the at least one barcode comprises an alpha code and a numerical code that, in combination, identify the customer.

52. The method of claim 49, wherein alerting the at least one medical laboratory of the at least one authorized laboratory test comprises providing the at least one medical laboratory with the at least one barcode of the delivered sample collection test kit.

53. The method of claim 44, wherein receiving authorization for the at least one requested laboratory test from the at least one first medical professional comprises assigning at least one test code to each authorized laboratory test.

54. The method of claim 44, wherein receiving authorization from the at least one first medical professional comprises, after receiving a request for the customer for the at least one laboratory test:
identifying a first medical professional to authorize the at least one requested laboratory test; and
transmitting the request and some portion of a medical history of the customer to the identified first medical professional.

55. The method of claim 44 further comprising at least one of:
receiving confirmation that the customer has registered the sample collection test kit in a customer account;
receiving confirmation that the customer has registered two unique identifiers associated with the sample collection test kit in the customer account;
receiving confirmation that the customer has activated the sample collection test kit; and
receiving confirmation that the at least one medical laboratory has received the activated sample collection test kit.

56. The method of claim 55, wherein receiving confirmation that the customer has activated the sample collection test kit comprises at least one of:
receiving patient medical history data from the customer; and
receiving a suitability questionnaire completed by the customer.

57. The method of claim 55, further comprising, after receiving confirmation that the customer has activated the sample collection test kit, attributing the customer to the registered two unique identifiers.

58. The method of claim 55, further comprising, after receiving confirmation that the customer has activated the sample collection test kit, attributing the customer to an assigned test code.

59. The method of claim 44 further comprising:
assigning a test code to each authorized sample collection test kit that has been delivered to the customer; and
matching laboratory test results with the customer using the assigned test code.

60. The method of claim 44, wherein facilitating the at least one second medical professional interpreting the results comprises transmitting the results of the at least one authorized laboratory test to the at least one second medical professional.

61. The method of claim 44, wherein facilitating the at least one second medical professional providing medical guidance based thereon comprises transmitting at least one of medical records of the customer, personal medical history of the customer, and at least one suitability questionnaire completed by the customer to the at least one second medical professional.

62. The method of claim 44, wherein facilitating the at least one second medical professional providing medical guidance based thereon comprises at least one of:
enabling the at least one second medical professional to communicate with the customer via at least one of a video conference via the online portal of the customer, a telephone conference via a customer's telephone, and a cellphone conference via a customer's cellphone; and
enabling the at least one second medical professional to transmit to the online portal of the customer at least one of: laboratory test results, an e-prescription, medical notes of the medical professional, and medical advice of the medical professional.
